(19) United States
(12) Patent Application Publication
(10) Pub. No.: US 2026/0263415 A1
(43) Pub. Date: Sep. 10, 2026

(54) Sulfonamides With Activity in Acute Kidney Injury and Fibrosis

(71) Applicant: University of Pittsburgh - Of the Commonwealth System of Higher Education, Pittsburgh, PA (US)

(72) Inventors: Donna Huryn, Philadelphia, PA (US); Neil Hukriede, Allison Park, PA (US)

(21) Appl. No.: 19/473,272

(22) PCT Filed: Apr. 11, 2024
(86) PCT No.: PCT/US2024/024065

§ 371 Date: Oct. 7, 2025

Related U.S. Application Data
(60) Provisional application No. 63/458,726, filed on Apr. 12, 2023.

(51) Int. Cl. A61K 31/381; A61K 31/18; A61P 13/12; C07C 311/03; C07D 333/60

(52) U.S. Cl. CPC A61K 31/381; A61K 31/18; A61P 13/12; C07C 311/03; C07D 333/60

(57) ABSTRACT

Sulfonamide compounds and sulfonamide-containing compositions are provided. The compounds and compositions are useful in methods of treatment of kidney injury and fibrosis.

### CROSS-REFERENCE TO RELATED APPLICATIONS

This application is the United States national phase of International Application No. PCT/US24/24065, filed Apr. 11, 2024, and claims priority to U.S. Provisional Patent Application, No. 63/458,726, filed Apr. 12, 2023, the disclosures of which are hereby incorporated by reference in their entireties.

### STATEMENT REGARDING FEDERAL FUNDING

This invention was made with government support under W81XWH-17-1-0610 awarded by the Defense Health Agency, Medical Research and Development Branch, and DK069403, and DK126122 awarded by the National Institutes of Health. The government has certain rights in the invention.

### BACKGROUND OF THE INVENTION

Compounds and compositions are provided with activity in treatment of kidney injury and fibrosis. The molecules provide an advance over previous compounds as they do not require a pro-drug for delivery.

Kidney injury, e.g., acute kidney injury (AKI) is remarkably common and has an unacceptably high mortality that has been unchanged for the last twenty years. AKI therapies that have been developed in experimental models when administered prior to the onset of injury have failed to show therapeutic benefit in humans. However, the kidney has an innate capacity to undergo epithelial regeneration following injury, suggesting that drugs that enhance this regenerative capacity are more likely to be of benefit when given after the onset of injury.

AKI is a multi-factorial disorder that occurs in approximately 7% of in-patients' hospital admissions. It is an independent predictor of in-hospital mortality. Severe AKI requiring renal replacement therapy occurs in 4% of critically ill patients and has 50% in-patient mortality. Long term studies in survivors of severe AKI indicate that approximately 12.5% become dialysis-dependent. There is an urgent need to develop effective therapies that will accelerate the rate of recovery following induction of renal injury.

Fibrosis results from the excessive accumulation of fibrous connective tissue in and around inflamed or damaged tissue. Fibrosis can lead to permanent scarring, organ malfunction and death, as with end-stage liver disease, cirrhosis, kidney disease, idiopathic pulmonary fibrosis (IPF), and heart failure. Collagen deposition is an important and reversible part of wound healing in normal tissue repair. However, it can result in an irreversible fibrotic response if the tissue injury is severe or repetitive, or if the wound-healing response becomes dysregulated. Many chronic autoimmune diseases lead to fibrosis, such as, without limitation, scleroderma, rheumatoid arthritis, Crohn's disease, ulcerative colitis, myelofibrosis, and systemic lupus erythematosus. Fibrosis also can influence tumor invasion and metastasis, chronic graft rejection, and the pathogenesis of many progressive myopathies. There are few effective treatments that effectively target fibrosis (See, e.g., Wynn T A, et al. Mechanisms of fibrosis: therapeutic translation for fibrotic disease. Nat Med. 2012 Jul. 6; 18(7):1028-40 for additional details regarding fibrosis, its causes, and associated diseases).

Effective compositions and treatments for AKI and fibrosis are needed.

### SUMMARY

A compound is provided. The compound having the exemplary structure (I):

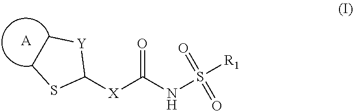

wherein:
   A is a (C5-C7)(hetero)aryl ring and/or a phenyl bioisostere;
   X is (C1-C7)divalent (hetero)alkyl;
   Y is methylene (—CH₂—) or dimethylene (—CH₂—CH₂—) forming a 5- or 6-member ring with 0, 1, or 2 double bonds, or is not present; and
   R₁ is (C1-C7)(hetero)alkyl, (C3-C7)(hetero)cycloalkyl, (C1-C3)(hetero)alkyl-(C3-C7)(hetero)cycloalkyl, (C1-C3)(hetero)alkyl-(C3-C7)aryl, (C1-C6)(hetero)alkoxyl, (C3-C7)(hetero)aryl-oxyl, or (hetero)aryl,
wherein A, X, or Y are, independently optionally substituted with one or more (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)(hetero)aryl-oxyl, (C3-C7)(hetero)aryl-amino, (C3-C7)(hetero)aryl-cyano, (C3-C7) (hetero)aryl-nitro, or (C3-C7) (hetero)aryl-halo groups,
   or a pharmaceutically-acceptable salt thereof. A composition also is provided comprising the compound and a pharmaceutically-acceptable excipient.

A method of treating kidney injury in a patient is provided. The method comprises administering to the patient a compound as described in the preceding paragraph, in an amount effective to treat kidney injury in the patient. A method of treating fibrosis, in a patient also is provided, comprising administering to the patient a compound as described in the preceding paragraph to the patient in an amount and dosage regimen effective to treat fibrosis, in the patient.

The following numbered clauses outline various aspects, embodiments, and/or examples of the present invention.
Clause 1. A compound is provided, having the structure (I):

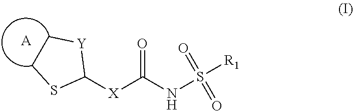

wherein:
   A is a (C5-C7)(hetero)aryl ring and/or a phenyl bioisostere;
   X is (C1-C7)divalent (hetero)alkyl; Y is methylene (—CH₂—) or dimethylene (—CH₂—CH₂—) forming a 5- or 6-member ring with 0, 1, or 2 double bonds, or is not present; and
   R₁ is (C1-C7)(hetero)alkyl, (C3-C7)(hetero)cycloalkyl, (C1-C3)(hetero)alkyl-(C3-C7)(hetero)cycloalkyl, (C1-C3)(hetero)alkyl-(C3-C7)aryl, (C1-C6)(hetero)alkoxyl, (C3-C7)(hetero)aryl-oxyl, or (hetero)aryl,
wherein A, X, or Y are, independently optionally substituted with one or more (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)(hetero)aryl-oxyl, (C3-C7)(hetero)aryl-amino, (C3-C7)(hetero)aryl-cyano, (C3-C7) (hetero)aryl-nitro, or (C3-C7) (hetero)aryl-halo groups,
   or a pharmaceutically-acceptable salt thereof.
Clause 2. The compound of clause 1, having the structure (II):

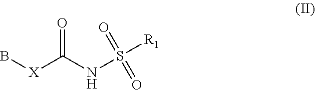

where:
B is:

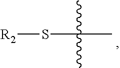

where R₂ is a C5-C7(hetero)aryl ring, or
a bicyclic heteroaryl group comprising a thiophene or thiopyran ring and a (C5-C7)aryl ring, such as a benzothiophenyl or benzothiopyranyl moiety;
X is (C1-C7)divalent (hetero)alkyl; and
R₁ is (C1-C7)(hetero)alkyl, (C3-C7)(hetero)cycloalkyl, (C1-C3)(hetero)alkyl-(C3-C7)(hetero)cycloalkyl, (C1-C3)(hetero)alkyl-(C3-C7)aryl, (C1-C6)(hetero)alkoxyl, (C3-C7)(hetero)aryl-oxyl, (hetero)aryl, or a bioisostere of any of the preceding;
   wherein B or X are, independently, optionally substituted with one or more (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)(hetero)aryl-oxyl, (C3-C7)(hetero)aryl-amino, (C3-C7)(hetero)aryl-cyano, (C3-C7)(hetero)aryl-nitro, (C3-C7)(hetero)aryl-halo groups, and with the exception of X, halo, or a bioisostere of any of the preceding, or a pharmaceutically-acceptable salt thereof.
Clause 3. The compound of clause 2, wherein a bicyclic heteroaryl group comprising a thiophene or thiopyran ring and an aryl ring.
Clause 4. The compound of clause 3, wherein the bicyclic heteroaryl group is a benzothiophenyl or benzothiopyranyl moiety.
Clause 5. The compound of clause 4, wherein B is

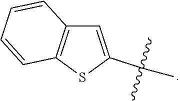 [SMILES: *c1cc2ccccc2s1]

Clause 6. The compound of clause 2, wherein B is

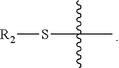

Clause 7. The compound of clause 6, wherein R₂ is phenyl, optionally substituted with one or more of (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, (C3-C7)aryl-amino, (C3-C7)aryl-cyano, (C3-C7)aryl-nitro, or (C3-C7)aryl-halo groups, or a bioisostere of any of the preceding.
Clause 8. The compound of any one of clauses 2-7, wherein X is alkyl.
Clause 9. The compound of clause 1, wherein Y is not present.
Clause 10. The compound of clause 1, having the structure:

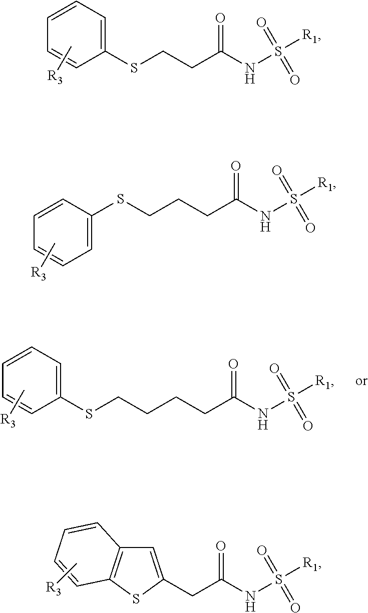

wherein R₃ is one or more of (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, (C3-C7)aryl-amino, (C3-C7)aryl-cyano, (C3-C7)aryl-nitro, or (C3-C7)aryl-halo groups, or a pharmaceutically acceptable salt thereof.
Clause 11. The compound of clause 1, having the structure:

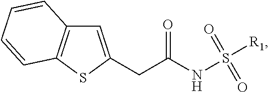

or a pharmaceutically acceptable salt thereof.
Clause 12. The compound of any one of clauses 1-11, wherein R₁ is
Clause 13. The compound of clause 1, having the structure:

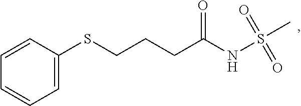 [SMILES: CS(=O)(=O)NC(=O)CCCSc1ccccc1]

or a pharmaceutically acceptable salt thereof.
Clause 14. The compound of clause 1, having the structure:

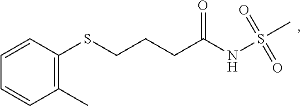 [SMILES: Cc1ccccc1SCCCC(=O)NS(C)(=O)=O]

or a pharmaceutically acceptable salt thereof.
Clause 15. The compound of the compound of clause 1, having the structure:

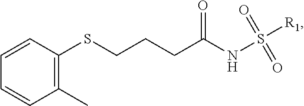

wherein R₁ is ethyl, cyclopropyl, n-propyl, isopropyl, or t-butyl, or a pharmaceutically acceptable salt thereof.
Clause 16. The compound of clause 1, having the structure:

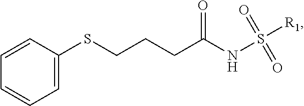

wherein R₁ is ethyl, cyclopropyl, n-propyl, isopropyl, or t-butyl.
Clause 17. The compound of clause 1 or 2, wherein X is substituted with one or two methyl groups.
Clause 18. The compound of clause 17, wherein R₁ is methyl, and R₂ is o-methyl phenyl.
Clause 19. The compound of clause 1, having the structure:

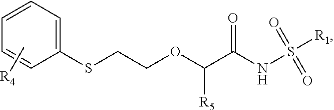

wherein, R₄ is one or more of H, (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, (C3-C7)aryl-amino, (C3-C7)aryl-cyano, (C3-C7)aryl-nitro, or (C3-C7)aryl-halo groups, and R₅ is (C1-C7)(hetero)alkyl or optionally substituted (C1-C7)(hetero)alkyl(hetero)aryl, optionally substituted with one or more of (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, (C3-C7)aryl-amino, (C3-C7)aryl-cyano, (C3-C7)aryl-nitro, or (C3-C7)aryl-halo groups, or a bioisostere of any of the preceding,
or a pharmaceutically acceptable salt thereof.
Clause 20. The compound of clause 19, wherein R₅ is methylphenyl.
Clause 21. The compound of clause 19 or 20, wherein R₄ is one or more of H or methyl.
Clause 22. The compound of clause 19, having the structure:

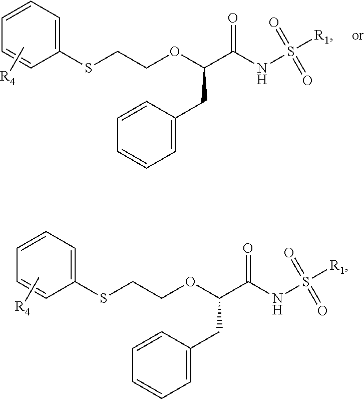

or a mixture thereof, and/or a pharmaceutically acceptable salt thereof.
Clause 23. The compound of clause 22, wherein R₄ is H.
Clause 24. The compound of clause 1 having a structure:

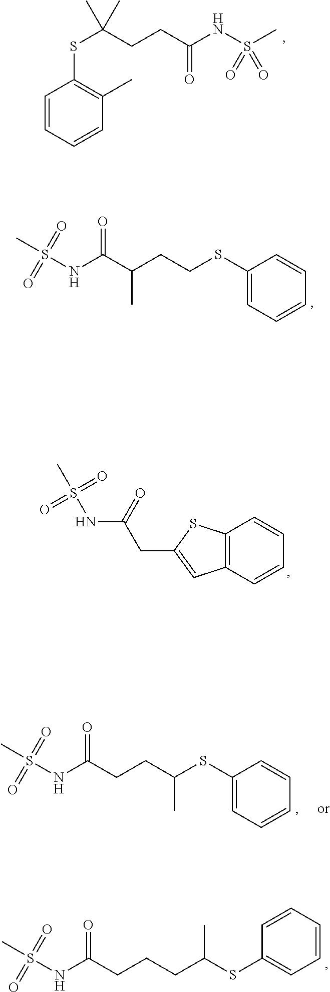

or a pharmaceutically acceptable salt thereof.
Clause 25. A composition comprising the compound of any one of clauses 1-24, and a pharmaceutically-acceptable excipient.
Clause 26. The composition of clause 25, comprising a compound having the structure:

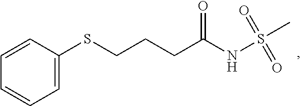 [SMILES: CS(=O)(=O)NC(=O)CCCSc1ccccc1]

or a pharmaceutically acceptable salt thereof; or

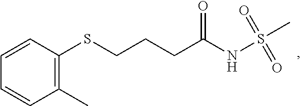 [SMILES: Cc1ccccc1SCCCC(=O)NS(C)(=O)=O]

or a pharmaceutically acceptable salt thereof.
Clause 27. A method of treating kidney injury in a patient, comprising administering to the patient a compound as described in any one of clauses 1-24, in an amount effective to treat kidney injury in the patient.
Clause 28. The method of clause 27, wherein the kidney injury is acute kidney injury.
Clause 29. The method of clause 27, wherein the kidney injury is related to trauma in the patient.
Clause 30. The method of clause 27, wherein the kidney injury is associated with: chronic kidney disease; decreased blood flow to the kidneys (e.g., due to blood or drug-induced kidney damage); blood or fluid loss; use of blood pressure medications; heart attack; heart disease; infection; liver failure; sepsis; use of NSAIDs (non-steroidal anti-inflammatory drugs) such as aspirin, ibuprofen, or naproxen sodium; severe allergic reaction (e.g., anaphylaxis); burns; dehydration; blood clots in the veins and arteries in and around the kidneys; cholesterol deposits that block blood flow in the kidneys; glomerulonephritis; inflammation of the glomeruli; hemolytic uremic syndrome; lupus; use of medications such as chemotherapy drugs, antibiotics, or dyes used during imaging tests; scleroderma; thrombotic thrombocytopenic purpura; toxins including, for example, alcohol, heavy metals or cocaine; rhabdomyolysis; tumor lysis syndrome; bladder cancer; blood clots in the urinary tract; cervical cancer; colon cancer; enlarged prostate (e.g., benign prostatic hyperplasia); kidney stones; nerve damage involving the nerves that control the bladder; or prostate cancer.
Clause 31. A method of treating fibrosis, in a patient comprising administering to the patient a compound as described in any one of clauses 1-24 to the patient in an amount and dosage regimen effective to treat fibrosis in the patient.
Clause 32. The method of clause 31, wherein the fibrosis is pulmonary fibrosis, such as idiopathic pulmonary fibrosis.
Clause 33. The method of clause 31, wherein the fibrosis is associated with: scleroderma; rheumatoid arthritis; Crohn's disease; ulcerative colitis; myelofibrosis; systemic lupus erythematosus; liver cirrhosis; non-alcoholic steatohepatitis; interstitial lung disease; acne; rosacea; kidney-, pancreas-, or heart-fibrosis.

### BRIEF DESCRIPTION OF THE DRAWINGS

FIG. 1 depicts the various compounds prepared as described in FIG. 2.

FIG. 2 provides an exemplary synthesis scheme for the compounds described herein.

FIGS. 3A and 3B provide structures and hazard ratios for exemplary compounds described herein.

FIGS. 4A and 4B provide structures for exemplary derivatives of UPHH207 (FIG. 4A) and UPHH231 (FIG. 4B).

FIG. 5 provides synthesis schemes and structures for additional compounds.

FIG. 6 is a Kaplan-Meier curve depicting, for compound 207, survival of zebrafish treated with 4 μM of compound 207.

FIG. 7—qPCR results for inflammatory and fibrosis markers (HAVCR1. HMOX1, IL6, CCL2, CCXCL1, COL1A1.) in human kidney organoids treated +/−with 25 μM hemin and +/−'207 at 0.2, 1 and 5 μM.

FIG. 8. Collagen 1A1 antibody staining of human kidney organoids treated +/−with 25 μM hemin and +/−'207 at 0.04, 0.2, 1 and 5 μM (left panel). Quantification of Collagen 1A1 antibody staining using 1-way Anova test, **=0.01 (right panel).

FIGS. 9A and 9B provide graphs showing plasma concentrations of compound 207 (FIG. 9A) and compounds 0020 and 186 (FIG. 9B), respectively).

FIG. 10 is a graph showing blood urea nitrogen (BUN) levels over time for mice treated with compound 207 (n=4).

FIG. 11 is a graph depicting and transdermal GFR (tGFR) 27 days after injury.

FIG. 12 is a graph depicting Sirius red staining of collagen in kidney sections 28 days after injury as a measure of fibrosis.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more.

As used herein, the term “comprising” is open-ended and may be synonymous with “including”, “containing”, or “characterized by”. As used herein, embodiments “comprising” one or more stated elements or steps also include but are not limited to embodiments “consisting essentially of” and “consisting of” these stated elements or steps.

A “moiety” (pl. “moieties”) is a part of a chemical compound, and includes groups, such as functional groups.

As used herein, “alkyl” refers to straight, branched chain, and/or cyclic hydrocarbon groups including, for example, from 1 to about 20 carbon atoms, for example and without limitation C1-C3, C1-C6, C1-C10 groups, for example and without limitation, straight, branched chain alkyl groups such as methyl, ethyl, propyl, butyl, pentyl, hexyl, heptyl, octyl, nonyl, decyl, undecyl, dodecyl, and the like. An alkyl group can be, for example, a C1, C2, C3, C4, C5, C6, C7, C8, C9, or C10 group that is substituted or unsubstituted. “lower alkyl” refers to C1-C6 alkyl. Non-limiting examples of straight alkyl groups include methyl, ethyl, propyl, butyl, pentyl, hexyl, heptyl, octyl, nonyl, and decyl. Branched alkyl groups comprise any straight alkyl group substituted with any number of alkyl groups. Non-limiting examples of branched alkyl groups include isopropyl, n-butyl, isobutyl, sec-butyl, and t-butyl. “Unsaturated alkyl” may comprise one or more, e.g., 1, 2, 3, 4, or 5, carbon-to-carbon double bonds and alternatively may be referred to as alkene or alkenyl, as described below. “Substituted alkyl” can include alkyl substituted at 1 or more (e.g., 1, 2, 3, 4, 5, 6, or more) positions, which substituents are attached at any available atom to produce a stable compound, with substitution as described herein. “Optionally substituted alkyl” refers to alkyl or substituted alkyl. “Halogen,” “halide,” and “halo” refers to —F, —Cl, —Br, and/or —I. “Alkylene” and “substituted alkylene” can include divalent alkyl and divalent substituted alkyl, respectively, including, without limitation, methylene, ethylene, trimethylene, tetramethylene, pentamethylene, hexamethylene, hepamethylene, octamethylene, nonamethylene, or decamethylene. “Optionally substituted alkylene” can include alkylene or substituted alkylene.

Non-limiting examples of cyclic alkyl groups include cyclopropyl, cyclobutyl, cyclopentyl, cyclohexyl, cycloheptlyl, and cyclooctyl groups. Cyclic alkyl groups also comprise fused-, bridged-, and spiro-bicycles and higher fused-, bridged-, and spiro-systems. A cyclic alkyl group can be substituted with any number of straight, branched, or cyclic alkyl groups. A cycloalkyl group may be attached via any atom. Cycloalkyl also contemplates fused rings where the cycloalkyl is fused to an aryl or hetroaryl ring. A cycloalkyl group can be unsubstituted or optionally substituted with one or more substituents as described herein below. “Cycloalkylene” refers to divalent cycloalkyl. The term “optionally substituted cycloalkylene” refers to cycloalkylene that is substituted with at least 1, 2 or 3 substituents, attached at any available atom to produce a stable compound, wherein the substituents are as described herein. A cycloalkylene may be formed by two “R groups” taken together, such as with “R₂ and R₃ taken together,” as referenced below.

“Alkene or alkenyl” can include straight, branched chain, or cyclic hydrocarbyl groups including, e.g., from 2 to about 20 carbon atoms having one or more, e.g., 1, 2, 3, 4, or 5, carbon-to-carbon double bonds, and may be referred to as “unsaturated alkyl”. The olefin or olefins of an alkenyl group can be, for example, E, Z, cis, trans, terminal, or exo-methylene. An alkenyl or alkenylene group can be, for example, a C2, C3, C4, C5, C6, C7, C8, C9, C10, C11, C12, C13, C14, C15, C16, C17, C18, C19, or C20 group that is substituted or unsubstituted. A halo-alkenyl group can be any alkenyl group substituted with any number of halogen atoms. “Substituted alkene” can include alkene substituted at 1 or more, e.g., 1, 2, 3, 4, or 5 positions, which substituents are attached at any available atom to produce a stable compound, with substitution as described herein. “Optionally substituted alkene” can include alkene or substituted alkene. Likewise, “alkenylene” can refer to divalent alkene. Examples of alkenylene include without limitation, ethenylene (—CH═CH—) and all stereoisomeric and conformational isomeric forms thereof. “Substituted alkenylene” can refer to divalent substituted alkene. “Optionally substituted alkenylene” can refer to alkenylene or substituted alkenylene.

Alkyne or “alkynyl” refers to a straight, branched chain, or cyclic unsaturated hydrocarbon having the indicated number of carbon atoms and at least one triple bond. The triple bond of an alkyne or alkynyl group can be internal or terminal. Examples of a (C2-C8)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below. An alkyne or alkynyl group can be, for example, a C2, C3, C4, C5, C6, C7, C8, C9, C10, C11, C12, C13, C14, C15, C16, C17, C18, C19, or C20 group that is substituted or unsubstituted. A halo-alkynyl group can be any alkynyl group substituted with any number of halogen atoms. The term “alkynylene” refers to divalent alkyne. Examples of alkynylene include without limitation, ethynylene, propynylene. “Substituted alkynylene” refers to divalent substituted alkyne.

“Carboxyl” or “carboxylic” refers to group having an indicated number of carbon atoms, where indicated, and terminating in a —C(O)OH group, thus having the structure —R—C(O)OH, where R is an unsubstituted or substituted divalent organic group that can include linear, branched, or cyclic hydrocarbons. Non-limiting examples of these include: C1-C8 carboxylic groups, such as ethanoic, propanoic, 2-methylpropanoic, butanoic, 2,2-dimethylpropanoic, pentanoic, etc. “Amine” or “amino” refers to group having the indicated number of carbon atoms, where indicated, and terminating in a —NH2 group, thus having the structure —R—NH₂, where R is an unsubstituted or substituted divalent organic group that, e.g., includes linear, branched, or cyclic hydrocarbons, and optionally comprises one or more heteroatoms. The term “alkylamino” refers to a radical of the formula —NHRₓ or —NRₓRₓ where each Rₓ is, independently, an alkyl radical as defined above. “Alkoxyl” or “alkoxy” refers to an —O-alkyl group, such as methoxyl, ethoxyl, propyloxyl, etc.

“Aryl,” alone or in combination refers to an aromatic ring system such as phenyl or naphthyl. “Aryl” also can include aromatic ring systems that are optionally fused with a cycloalkyl ring. A “substituted aryl” is an aryl that is independently substituted with one or more substituents attached at any available atom to produce a stable compound, wherein the substituents are as described herein. The substituents can be, for example, hydrocarbyl groups, alkyl groups, alkoxy groups, and halogen atoms. “Optionally substituted aryl” refers to aryl or substituted aryl. An aryloxy group can be, for example, an oxygen atom substituted with any aryl group, such as phenoxy. An arylalkoxy group can be, for example, an oxygen atom substituted with any aralkyl group, such as benzyloxy. “Arylene” denotes divalent aryl, and “substituted arylene” refers to divalent substituted aryl. “Optionally substituted arylene” refers to arylene or substituted arylene. A “polycyclic aryl group” and related terms, such as “polycyclic aromatic group” refers to a group composed of at least two fused aromatic rings. “Heteroaryl” or “hetero-substituted aryl” refers to an aryl group substituted with one or more heteroatoms, such as N, O, P, and/or S. Examples of heteroaryl groups include, but are not limited to, thienyl, furyl, pyridyl, oxazolyl, quinolyl, thiophenyl, thiopyranyl, benzothiophenyl, benzothiopyranyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

Non-limiting examples of optionally-substituted benzothiophenyl and benzothiopyranyl groups (e.g., as “A” in structure (II), below, or where Y is methylene or dimethylene, respectively, in structure (I), below) include:

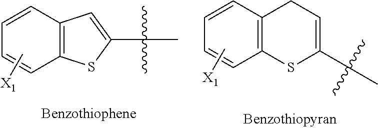

where X₁ independently may be H or a substituent, for example (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl (—O—(C3-C7)aryl), or halo groups.

Terms combining the foregoing refer to any suitable combination of the foregoing, such as arylalkenyl, arylalkynyl, heteroarylalkyl, heteroarylalkenyl, heteroarylalkynyl, heterocyclylalkyl, heterocyclylalkenyl, heterocyclylalkynyl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, and alkynylhereroaryl. As an example, “arylalkylene” refers to a divalent alkylene wherein one or more hydrogen atoms in an alkylene group is replaced by an aryl group, such as a (C3-C8)aryl group. Examples of (C3-C8)aryl-(C1-C6)alkylene groups include without limitation 1-phenylbutylene, phenyl-2-butylene, I-phenyl-2-methylpropylene, phenylmethylene, phenylpropylene, and naphthylethylene. The term “(C3-C8)cycloalkyl-(C1-C6)alkylene” refers to a divalent alkylene wherein one or more hydrogen atoms in the C1-C6 alkylene group is replaced by a (C3-C8)cycloalkyl group. Examples of (C3-C8)cycloalkyl-(C1-C6)alkylene groups include without limitation 1-cycloproylbutylene, cycloproyl-2-butylene, cyclopentyl-1 phenyl-2-methylpropylene, cyclobutylmethylene, and cyclohexylpropylene.

Terms not referenced above may take on a meaning as is understood to those of ordinary skill in the chemical and pharmaceutical arts.

A bioisostere in reference to a group or moiety, refers to structural motifs that express similar biological properties without the fundamental stipulation that they present a similar shape and size or express close physicochemical attributes, as would be expected of functionalities that share an isosteric relationship. Phenyl bioisosteres represent common isosteric substitutions in medicinal chemistry. Exemplary potential bioisosteres of monosubstituted (terminal) and disubstituted (para-, meta-, ortho-) benzene rings, as they are understood in the medicinal chemistry arts are described in detail in (Subbaiah M A M, Meanwell N A. Bioisosteres of the Phenyl Ring: Recent Strategic Applications in Lead Optimization and Drug Design. J Med Chem. 2021 Oct. 14; 64(19):14046-14128, see, e.g., FIG. 38 thereof). As with phenyl bioisosteres, suitable bioisosteric substitutions of other groups are broadly-known to those of ordinary skill in the medicinal chemistry arts.

As used herein, the term “patient” or “subject” refers to members of the animal kingdom including but not limited to human beings and “mammal” refers to all mammals, including, but not limited to human beings.

As used herein, the “treatment” or “treating” of a patient means administration to a patient by any suitable dosage regimen, procedure and/or administration route of a composition, device, or structure with the object of achieving a beneficial or desirable clinical/medical end-point, including but not limited to, preventing, reducing, and/or eliminating any symptom of acute kidney injury or fibrosis. An amount of any agent, administered by any suitable route, effective to treat a patient is an amount capable of preventing, reducing, and/or eliminating any symptom of acute kidney injury or fibrosis. Any suitable clinical marker may be used to determine efficacy of treatment, including, without limitation, improved survival, improved kidney function, or reduced fibrosis or a biological marker of any of the preceding. Clinical assay results can be said to “normalize” when such clinical markers approach or enter a normal or healthy range for a patient.

The compositions described herein can be administered by any effective route, such as parenteral, e.g., intravenous, intramuscular, subcutaneous, intradermal, perfusion of organ or tissue, application to organ or tissue, etc., formulations of which are described below and in the below-referenced publications, as well as are broadly-known to those of ordinary skill in the art.

Suitable dosage forms may include single-dose, or multiple-dose vials or other containers, such as medical syringes, containing a composition comprising an active ingredient, such as a compound as described herein.

Drug products, or pharmaceutical compositions comprising an active agent (e.g., drug), for example, a compound as described herein, may be prepared by any method known in the art of pharmacy, for example, by bringing into association the active ingredient with the carrier(s) or excipient(s). As used herein, a “pharmaceutically acceptable excipient”, “carrier” or “pharmaceutically acceptable carrier” includes any solvents, dispersion media, coatings, antibacterial and antifungal agents, isotonic and absorption delaying agents, and the like that are physiologically compatible. Examples of pharmaceutically acceptable excipients include one or more of water, saline, phosphate buffered saline, dextrose, glycerol, ethanol, and the like, as well as combinations thereof. In many cases, it may be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives, or buffers, which enhance the shelf life or effectiveness of the active agent. The active agent may be prepared with a carrier that will protect the compound against rapid release, such as a controlled release formulation, including implants, transdermal patches, and microencapsulated delivery systems. Biodegradable, biocompatible polymers can be used in delivery systems, such as ethylene vinyl acetate, polyanhydrides, polyglycolic acid, collagen, polyorthoesters, and polylactic acid. Further to the above, non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts. Methods for the preparation of such formulations are broadly-known to those skilled in the art.

Additionally, active agent-containing compositions may be in a variety of forms. The preferred form depends on the intended mode of administration and therapeutic application, which will in turn dictate the types of carriers/excipients. Suitable forms include, but are not limited to, liquid, semi-solid and solid dosage forms.

Pharmaceutical formulations adapted for oral administration may be presented, for example and without limitation, as discrete units such as capsules or tablets; powders or granules; solutions or suspensions in aqueous or non-aqueous liquids; edible foams or whips; or oil-in-water liquid emulsions or water-in-oil liquid emulsions. In certain embodiments, the active agent may be contained in a formulation such that it is suitable for oral administration, for example, by combining the active agent with an inert diluent or an assimilable edible carrier. The active agent (and other ingredients, if desired) may also be enclosed in a hard- or soft-shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Pharmaceutical formulations adapted for topical administration may be formulated, for example and without limitation, as ointments, creams, suspensions, lotions, powders, solutions, pastes, gels, sprays, aerosols, or oils. Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents, and other suitable additives. Pharmaceutically acceptable organic or inorganic excipients suitable for non-parenteral administration which do not deleteriously react with nucleic acids can be used.

Pharmaceutical formulations adapted for nasal administration wherein the carrier is a solid include a coarse powder having a particle size, for example, in the range 20 to 500 microns which is administered in the way snuff is taken, e.g., by rapid inhalation through the nasal passage from a container of the powder held close to the nose. Suitable formulations wherein the carrier is a liquid, for administration as a nasal spray or as nasal drops, include aqueous or oil solutions of the active ingredient.

Pharmaceutical formulations adapted for administration by inhalation include, without limitation, fine particle dusts or mists which may be generated by means of various types of metered dose pressurized aerosols, nebulizers, or insufflators. In the context of delivery of the active agents described herein by inhalation, inhalation drug products, such as metered-dose inhalers, as are broadly-known in the pharmaceutical arts, are used. Metered dose inhalers are configured to deliver a single dose of an active agent per actuation, though multiple actuations may be needed to effectively treat a given patient.

Pharmaceutical formulations adapted for parenteral administration include aqueous and non-aqueous sterile injection solutions which may contain, for example and without limitation, anti-oxidants, buffers, bacteriostats, lipids, liposomes, emulsifiers, also suspending agents and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules, and tablets.

Other than living, cellular therapies, therapeutic compositions may be sterile and stable under the conditions of manufacture and storage. For example, sterile injectable solutions may be prepared by incorporating the active agent in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of the active ingredient plus any additional desired ingredient from a previously sterile-filtered solution thereof. The proper fluidity of a solution can be maintained, for example, using a coating such as lecithin, by the maintenance of the required particle size in the case of dispersion and using surfactants. Prolonged absorption of injectable compositions can be brought about by including in the composition an agent that delays absorption, for example, monostearate salts and gelatin.

The compounds described herein may be complexed with a cyclodextrin. Cyclodextrins are compounds that have found substantial recognition as excipients (e.g., as carriers, vehicles, etc.) in the pharmaceutical field, for example, in oral and intravenous dosage forms. Cyclodextrins are able form non-covalent inclusion complexes and/or aggregates in solution with poorly soluble drugs, for example, BCS Class II and IV drugs (high or low intestinal permeability, respectively, but low solubility in both instances). Cyclodextrins are cyclic oligosaccharides having a hydrophilic outer surface and a lipophilic central cavity. They consist of α-1,4-linked α-D-glucopyranose units. Naturally-occurring cyclodextrins include α-, β- and γ-cyclodextrins, with 6, 7, and 8 glucopyranose units, respectively. The natural cyclodextrins can be used orally or topically, but natural β-cyclodextrin and γ-cyclodextrin cannot be used parenterally. A number of cyclodextrin derivatives have been formulated with various usefulness in different administrative routes. Common, non-limiting examples of cyclodextrin derivatives include hydroxypropyl-β-cyclodextrin (e.g., 2-hydroxypropyl-β-cyclodextrin), hydroxypropyl-γ-cyclodextrin (e.g., 2-hydroxypropyl-γ-cyclodextrin), hydroxyethyl-β-cyclodextrin, randomly methylated β-cyclodextrin, methyl-β-cyclodextrin, dimethyl-β-cyclodextrin, permethylated β-cyclodextrin, sulfobutylether β-cyclodextrin (e.g., sodium salt), sulfobutyl-γ-cyclodextrin, branched cyclodextrin (e.g., glucosyl-β-cyclodextrin or maltosyl-β-cyclodextrin, e.g., 6-O-maltosyl-β-cyclodextrin or glucosyl-β-cyclodextrin) and randomly-acetylated amorphous-β-cyclodextrin. Cyclodextrins may be complexed with a drug as inclusion complexes (included) in a solution in a 1:1 molar ratio, though increased or decreases relative amounts of the drug or cyclodextrin may be used during formulation in order to drive the reaction. Where the drug is aggregated instead of included within the cyclodextrin, an excess of cyclodextrin may be utilized. It should be recognized that the inclusion or aggregation process can be optimized, including manipulation of relative cyclodextrin-to-active ingredient ratios to obtain optimal solubility and bioavailability or other desirable features of the end-product. See, e.g., Loftsson et al. “Self-Association of Cyclodextrins and Cyclodextrin Complexes” J. Pharm. Sci. 93(5):1091-1099 (2004); Loftsson et al. “Cyclodextrins in Drug Delivery” Expert. Opin. Drug Deliv. 2:335-351 (2005); Brewster et al. “Cyclodextrins as Pharmaceutical Solubilizers” Advanced Drug Delivery Reviews 59:645-666 (2007); and Rasheed et al., “Cyclodextrins as Drug Carrier Molecule: A review” Sci. Pharm. 76:567-598 (2008) for their description of cyclodextrins and uses thereof in the pharmaceutical arts. As used herein, “a cyclodextrin” or “cyclodextrins” refer not only to naturally-occurring α-, β- and γ-cyclodextrins, but to cyclodextrin derivatives, including, but not limited to those mentioned above. Likewise “α-cyclodextrin(s)”, “β-cyclodextrin(s)” and “γ-cyclodextrins” refer both to the naturally-occuring cyclodextrin and to cyclodextrin derivatives (e.g., “a β-cyclodextrin” includes both β-cyclodextrin and β-cyclodextrin derivatives, such as, without limitation, hydroxypropyl-β-cyclodextrin, hydroxyethyl-β-cyclodextrin, randomly methylated β-cyclodextrin, methyl-β-cyclodextrin, dimethyl-β-cyclodextrin, permethylated β-cyclodextrin, sulfobutylether β-cyclodextrin, branched β-cyclodextrin, etc.).

The formulation may be a liposome, lipid nanoparticle, drug-loaded extracellular vesicle, or multiphase (a liquid comprising more than one phase, such as oil in water, water in oil, liposomes or multi-lamellar structures) composition. Multi-phase systems, including liposomes, are prevalent in the pharmaceutical arts. In the case of a liposome, the drug product might comprise a phospholipid, a non-ionic detergent, and a cationic lipid, such as a composition comprising a phosphatidyl choline, a non-ionic surfactant, and a quaternary ammonium salt of a lipid-substituted D or L glutamic acid or aspartic acid, and an aqueous solvent. The liposomes or multiphase liquids and the ingredients thereof are pharmaceutically acceptable. They are typically formulated using an aqueous solvent, such as water, normal saline or PBS.

Phospholipids include any natural or synthetic diacylglyceryl phospholiopid (such as phosphatidyl choline, phosphotidylethanolamine, phosphotidylserine, phosphatidylinositol, phosphatidylinositol phosphate, etc.) and phosphosphingolipid that can form self-assembling liposomes. In one example, the phospolipid is a phosphatidyl choline, a compound that comprises a choline head group, glycerophosphoric acid and fatty acid. Phosphatidyl choline can be obtained from eggs, soy, or any suitable source and can be synthesized.

A nonionic surfactant is a surfactant containing no charged groups. Nonionic surfactants comprise a hydrophilic head group and a lipophilic tail group, such as a single- or double-lipophilic chain surfactant. Examples of lipophilic tail groups include lipophilic saturated or unsaturated alkyl groups (fatty acid groups), steroidal groups, such as cholesteryl, and vitamin E (e.g., tocopheryl) groups, such as a polysorbate (a polyoxyethylene sorbitan), for example, Tween 20, 40, 60, or 80. More broadly, non-ionic surfactants include: glyceryl esters, including mono-, di- and tri-glycerides; fatty alcohols; and fatty acid esters of fatty alcohols or other alcohols, such as propylene glycol, polyethylene glycol, sorbitan, sucrose and cholesterol.

A cationic lipid is a compound having a cationic head and a lipophilic tail. Included are cationic lipids that are quaternary ammonium salts, such as quaternary ammonium salts of lipid-substituted D and L glutamic acid or aspartic acid, such as glutamic acid dialkyl amides, including, for example, L-glutamic acid-1,5,-dioleyl amide. Other commercially-available examples of cationic lipids (e.g., available from Avanti Polar Lipids) include DC-Cholesterol (3β-[N-(N′,N′-dimethylaminoethane)-carbamoyl]cholesterol hydrochloride), DOTAP (e.g., 1,2-dioleoyl-3-trimethylammonium-propane (chloride salt)), DODAP (e.g., 1,2-dioleoyl-3-dimethylammonium-propane), DDAB (e.g., Dimethyldioctadecylammonium (Bromide Salt)), ethyl-PC (e.g., 1,2-dilauroyl-sn-glycero-3-ethylphosphocholine (chloride salt)) and DOTMA (e.g., 1,2-di-O-octadecenyl-3-trimethylammonium propane (chloride salt)).

The ratio of ingredients (phospholipid:nonionic surfactant:cationic lipid) can vary greatly, so long as a useful multilamellar structure is obtained that is able to deliver the active agents described herein. Further, each different combination of ingredients might have different optimal ratios. The ability to determine optimal ratios does not require undue experimentation because the ability of any formulation to deliver the active agent is readily tested as described herein, and as is generally known in the pharmaceutical arts. Liposome and multilamellar structures are common delivery vehicles for active agents and their manufacture, physical testing and biological assays to determine effectiveness are well-known. Useful phospholipid:nonionic surfactant:cationic lipid ratios include, for example: from 0.1-10:0.1-10:0.1-10 (w/w), and in certain instances the nonionic surfactant:cationic lipid (w/w) ratio is approximately the same and/or the phospholipid constituent is from 2 to 10 times (w/w) that of the nonionic surfactant and cationic lipid.

Antibody-drug conjugates, where a targeting antibody is reversibly bound to a drug, or is incorporated into a complex with the drug, such as a vesicular (e.g., lipid-mediated) drug delivery vehicle or lipid nanoparticle, may be employed to target the drug to a specific tissue or organ in a patient (see, e.g., Fu, Z, et al. Antibody drug conjugate: the “biological missile” for targeted cancer therapy. Signal Transduct Target Ther. 2022 Mar. 22; 7(1):93; Marques A C, et al. Lipid Nanoparticles Functionalized with Antibodies for Anticancer Drug Therapy. Pharmaceutics. 2023 Jan. 8; 15(1):216; and Topping L M, et al. Targeting Extracellular Vesicles to the Arthritic Joint Using a Damaged Cartilage-Specific Antibody. Front Immunol. 2020 Feb. 14; 11:10).

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single or multiple injection, tablet or capsule, or metered doses from a metered-dose inhaler, effective to achieve a determinable end-point. The “amount effective” is preferably safe—at least to the extent the benefits of treatment outweighs the detriments and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A therapeutically effective amount is also one in which any toxic or detrimental effects of the active agent are outweighed by the therapeutically beneficial effects. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). For example, a single bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc. The dosage may be proportionally reduced or increased as indicated by the exigencies of the therapeutic situation. In some instances, it may be especially advantageous to formulate parenteral or inhaled compositions in dosage unit form for ease of administration and uniformity of dosage. The specification for the dosage unit forms of the invention may be dictated by and directly dependent on (a) the unique characteristics of the active compound and the particular therapeutic or prophylactic effect to be achieved, and (b) the limitations inherent in the art of compounding such an active compound for the treatment of sensitivity in individuals.

The compound may be administered locally or topically at a site of wound, graft, or fibrotic lesion to prevent or treat inflammation, fibrosis, or scarring. Topical administration includes ocular delivery and dosage forms. In a patient with pulmonary fibrosis or other inflammatory conditions of the lungs and/or respiratory tract, the compound may be administered locally, e.g., by spray, nebulization, aerosolization, inhalation, or by bronchoalveolar lavage, or systemically, for example intravenously.

The compound may be administered to a patient systemically for treating kidney injury, treating acute kidney injury, improving kidney function, inhibiting a histone deacetylase in a cell, expanding renal progenitor cells and/or stimulating kidney repair in cells in vitro, ex vivo or in vivo (in a patient), and/or preventing fibrosis, e.g., fibrotic activity. Compositions also are provided for delivery of the compounds to a patient. Also provided are methods for treating kidney injury, treating acute kidney injury, improving kidney function, inhibiting a histone deacetylase in a cell, expanding renal progenitor cells and/or stimulating kidney repair in cells in vitro, ex vivo or in vivo (in a patient) comprising contacting the cells with, or administering to a patient and amount of one or more of the compounds effective to improve kidney function in a patient, inhibit a histone deacetylase in a cell, expand renal progenitor cells and/or stimulate kidney repair in cells. Therefore, provided are in vitro (including ex vivo) or in vivo (in a patient) methods. Efficacy of the compounds is demonstrated below. The compound described herein may be administered in any manner that is effective for treating kidney injury, treating acute kidney injury, improving kidney function, inhibiting a histone deacetylase in a cell, expanding renal progenitor cells and/or stimulating kidney repair in cells in a patient. The compounds described herein also may be administered in any manner that is effective to treat fibrosis or to reduce or prevent fibrotic activity. Examples of delivery routes include, without limitation: topical, for example, epicutaneous, inhalational, enema, ocular, otic and intranasal delivery; enteral, for example, orally, by gastric feeding tube or swallowing, and rectally; and parenteral, such as, intravenous, intraarterial, intramuscular, intracardiac, subcutaneous, intraosseous, intradermal, intrathecal, intraperitoneal, transdermal, iontophoretic, transmucosal, epidural and intravitreal. For treatment of kidney injury, acute kidney injury, stimulating kidney repair in cells, improving kidney function, inhibiting a histone deacetylase in a cell, and/or expanding renal progenitor cells oral or intravenous approaches may be employed.

As indicated above, fibrosis can lead to permanent scarring, organ malfunction and death, as with end-stage liver disease, cirrhosis, kidney disease, idiopathic pulmonary fibrosis (IPF), and heart failure. Collagen deposition is an important and reversible part of wound healing in normal tissue repair. However, it can result in an irreversible fibrotic response if the tissue injury is severe or repetitive, or if the wound-healing response becomes dysregulated. Many chronic autoimmune diseases lead to fibrosis, such as, without limitation, scleroderma, rheumatoid arthritis, Crohn's disease, ulcerative colitis, myelofibrosis, and systemic lupus erythematosus. Fibrosis also can influence tumor invasion and metastasis, chronic graft rejection, and the pathogenesis of many progressive myopathies. For treatment of fibrosis, the compounds described herein may be administered by any suitable route and dosage regimen. For example, in the case of wound healing and scar prevention, the compound may be formulated as a topical formulation, such as a cream, ointment, tincture, spray, or drops (e.g., for optic or otic topical use). For example, in the case of gastrointestinal wound healing, e.g., for prevention of scarring or strictures in surgical patients or patients with ulcers or inflammatory bowel disease, as in Crohn's disease or ulcerative colitis, the composition may be administered orally or via a suppository, or systemically, such as parenterally, e.g., by subcutaneous, intramuscular, intravenous, or intraperitoneal delivery routes. Other appropriate delivery routes may be utilized for specific diseases, such as intrathecally for treatment or prevention of epidural fibrosis.

In aspects, pharmaceutically acceptable salts or hydrates of any of the compounds described herein are provided and are used in the methods described herein. Pharmaceutically acceptable salt forms or hydrates of the compounds described herein may be prepared by conventional methods known in the pharmaceutical arts, for use in human or veterinary drug products. For example and without limitation, where a compound comprises a carboxylic acid group, a suitable salt thereof may be formed by reacting the compound with an appropriate base to provide the corresponding base addition salt. Non-limiting examples include: alkali metal hydroxides, such as potassium hydroxide, sodium hydroxide and lithium hydroxide; alkaline earth metal hydroxides, such as barium hydroxide and calcium hydroxide; alkali metal alkoxides, such as potassium ethanolate and sodium propanolate; and various organic bases such as piperidine, diethanolamine, and N-methylglutamine.

Acid and base addition salts may be prepared by contacting the free base form with a sufficient amount of a desired acid or base to produce the salt in a manner known in the art. The free base may be regenerated by contacting the salt form with a base or acid (depending on the nature of the salt) and isolating the free base. The free base forms differ from their respective salt forms somewhat in certain physical properties such as solubility in polar solvents, but otherwise the salts are equivalent to their respective free base forms for purposes described herein.

Compounds comprising basic nitrogen-containing groups may be quaternized with such agents as C₁₋₄ alkyl halides, such as methyl, ethyl, iso-propyl and tert-butyl chlorides, bromides and iodides; C₁₋₄ alkyl sulfate such as dimethyl, diethyl and diamyl sulfates; C₁₀₋₁₈ alkyl halides, such as decyl, dodecyl, lauryl, myristyl and stearyl chlorides, bromides and iodides; and aryl- C₁₋₄ alkyl halides, such as benzyl chloride and phenethyl bromide. Such salts permit the preparation of both water-soluble and oil-soluble compounds.

Non-limiting examples of pharmaceutically-acceptable base salts include: aluminum, ammonium, calcium, copper, ferric, ferrous, lithium, magnesium, manganic, manganous, potassium, sodium, and zinc salts. Salts derived from pharmaceutically acceptable organic non-toxic bases include, without limitation: salts of primary, secondary, and tertiary amines, substituted amines including naturally occurring substituted amines, cyclic amines, and basic ion exchange resins, such as arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

Acid addition salts may be prepared by treating a compound with pharmaceutically acceptable organic and inorganic acids, including, without limitation: hydrohalides, such as hydrochloride, hydrobromide, hydroiodide; other mineral acids and their corresponding salts such as sulfates, nitrates, and phosphates; alkyl- and mono-arylsulfonates, such as ethanesulfonate, toluenesulfonate, and benzenesulfonate; and other organic acids and their corresponding salts, such as acetate, tartrate, maleate, succinate, citrate, benzoate, salicylate, and ascorbate.

Non-limiting examples of pharmaceutically-acceptable acid salts include: acetate, adipate, alginate, arginate, aspartate, benzoate, besylate (benzenesulfonate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate, galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate, and phthalate.

Multiple salts forms are also considered to be pharmaceutically-acceptable salts. Common, non-limiting examples of multiple salt forms include: bitartrate, diacetate, difumarate, dimeglumine, diphosphate, disodium, and trihydrochloride. Hydrates and esters of the described compounds also may be produced by known methods.

As used herein, unless indicated otherwise, for instance in a structure, all compounds and/or structures described herein comprise all possible stereoisomers, individually or mixtures thereof. The compound and/or structure may be an enantiopure preparation consisting essentially of an (−) or (+) enantiomer of the compound, or may be a mixture of enantiomers in either equal (racemic) or unequal proportions.

Therapeutic/pharmaceutical compositions are prepared in accordance with acceptable pharmaceutical procedures. Any of the compounds described herein may be compounded or otherwise manufactured into a suitable composition for use, such as a pharmaceutical dosage form or drug product in which the compound is an active ingredient. According to one example, the drug product described herein is an oral tablet, capsule, caplet, liquid-filled or gel-filled capsule, etc. Compositions may comprise a pharmaceutically acceptable carrier, or excipient. An excipient is an inactive substance used as a carrier for the active ingredients of a medication. Although “inactive” excipients may facilitate and aid in increasing the delivery, stability, or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

A compound is provided, having the structure (I):

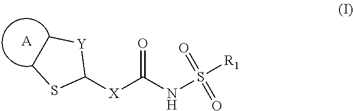

wherein:
   A is a (C5-C7)(hetero)aryl ring and/or a phenyl bioisostere;
   X is (C1-C7)divalent (hetero)alkyl;
   Y is methylene (—CH₂—) or dimethylene (—CH₂—CH₂—) forming a 5- or 6-member ring with 0, 1, or 2 double bonds, or is not present; and
   R₁ is (C1-C7)(hetero)alkyl, (C3-C7)(hetero)cycloalkyl, (C1-C3)(hetero)alkyl-(C3-C7)(hetero)cycloalkyl, (C1-C3)(hetero)alkyl-(C3-C7)aryl, (C1-C6)(hetero)alkoxyl, (C3-C7)(hetero)aryl-oxyl, or (hetero)aryl,
wherein A, X, or Y are, independently optionally substituted with one or more (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, (C3-C7)aryl-amino, (C3-C7)aryl-cyano, (C3-C7)aryl-nitro, or (C3-C7)aryl-halo groups,
   or a pharmaceutically-acceptable salt thereof.

The compound may have the structure (II):

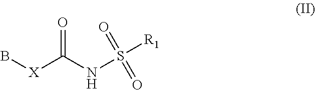

where:
B is:

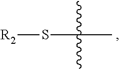

where R₂ is a C5-C7(hetero)aryl ring, or
a bicyclic heteroaryl group comprising a thiophene or thiopyran ring and a (C5-C7)aryl ring, such as a benzothiophenyl or benzothiopyranyl moiety;
X is (C1-C7)divalent (hetero)alkyl; and
R₁ is (C1-C7)(hetero)alkyl, (C3-C7)(hetero)cycloalkyl, (C1-C3)(hetero)alkyl-(C3-C7)(hetero)cycloalkyl, (C1-C3)(hetero)alkyl-(C3-C7)aryl, (C1-C6)(hetero)alkoxyl, (C3-C7)(hetero)aryl-oxyl, or halo, or a bioisostere of any of the preceding;
   wherein B or X are, independently, optionally substituted with one or more (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7) (hetero)aryl-oxyl, (C3-C7) (hetero)aryl-amino, (C3-C7) (hetero)aryl-cyano, (C3-C7) (hetero)aryl-nitro, (C3-C7) (hetero)aryl-halo groups, and with the exception of X, halo, or a bioisostere of any of the preceding, or a pharmaceutically-acceptable salt thereof.

The compound may have the structure:

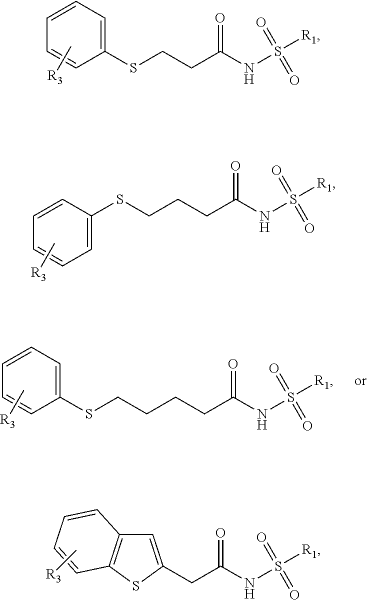

wherein R₃ is one or more of (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, or halo groups, or a pharmaceutically acceptable salt thereof.

The compound may have the structure:

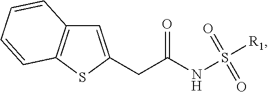

or a pharmaceutically acceptable salt thereof.

In examples, in structures (I) and (II), X may be substituted with one or two methyl groups. R₁ in any of the preceding may be methyl. In one example, in structure (II), R₁ may be methyl and R₂ may be o-methyl phenyl.

The compound may have the structure:

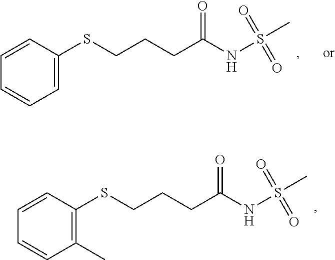 [SMILES: CS(=O)(=O)NC(=O)CCCSc1ccccc1 ; Cc1ccccc1SCCCC(=O)NS(C)(=O)=O]

or a pharmaceutically acceptable salt thereof.

The compound may have the structure:

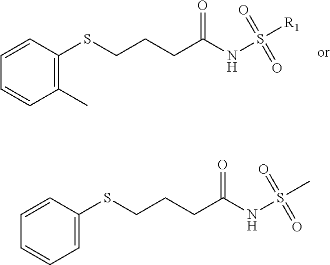

wherein R₁ is ethyl, cyclopropyl, n-propyl, isopropyl, or t-butyl, or a pharmaceutically acceptable salt thereof.

The compound may have the structure:

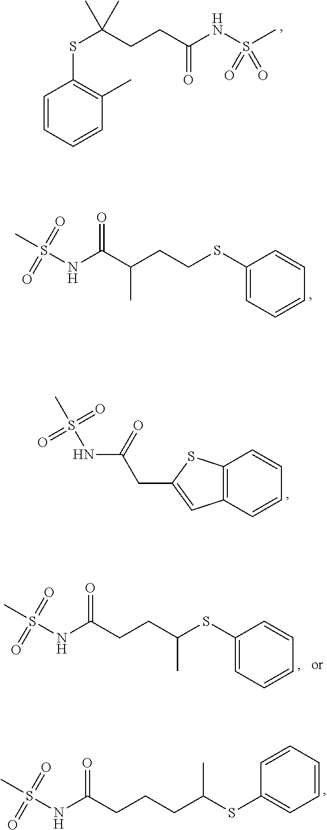

or a pharmaceutically acceptable salt thereof.

The compound may have the structure:

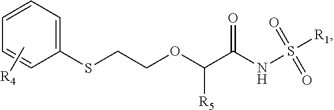

wherein, R₄ is one or more of H, (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, (C3-C7)aryl-amino, (C3-C7)aryl-cyano, (C3-C7)aryl-nitro, or (C3-C7)aryl-halo groups, and R₅ is (C1-C7)(hetero)alkyl or (C1-C7)(hetero)alkyl(hetero)aryl, optionally, either a bioisostere thereof, or substituted with one or more of (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, (C3-C7)aryl-amino, (C3-C7)aryl-cyano, (C3-C7)aryl-nitro, or (C3-C7)aryl-halo groups, or a pharmaceutically acceptable salt thereof. The compound may have the structure:

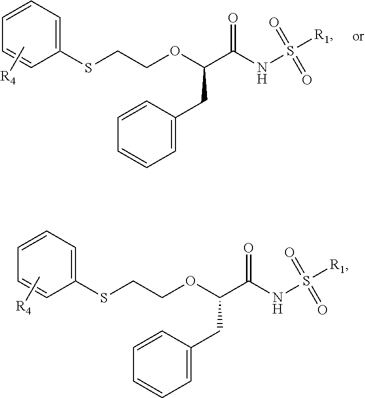

or a mixture thereof, and/or a pharmaceutically acceptable salt thereof, or may have the structure:

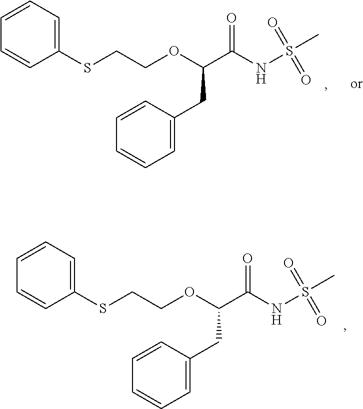 [SMILES: CS(=O)(=O)NC(=O)[C@@H](Cc1ccccc1)OCCSc1ccccc1 ; CS(=O)(=O)NC(=O)[C@H](Cc1ccccc1)OCCSc1ccccc1]

or a mixture thereof, and/or a pharmaceutically acceptable salt thereof.

A composition is provided comprising any of the preceding compounds and a pharmaceutically-acceptable excipient. The composition may comprise a compound having the structure:

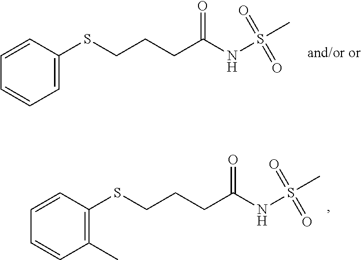 [SMILES: CS(=O)(=O)NC(=O)CCCSc1ccccc1 ; Cc1ccccc1SCCCC(=O)NS(C)(=O)=O]

or a pharmaceutically acceptable salt thereof.

Provided herein is a method of treating a kidney injury in a patient, where the kidney injury results from any disease or injury, such as administering to a patient an effective amount of a compound as described herein, administered in an amount effective to treat kidney injury in the patient. The injury may be acute kidney injury, and may be related to trauma or may be associated with any form of kidney disease or damage, such as, without limitation, associated with chronic kidney disease; decreased blood flow to the kidneys (e.g., due to blood or drug-induced kidney damage); blood or fluid loss; use of blood pressure medications; heart attack; heart disease; infection; liver failure; sepsis; use of NSAIDs (non-steroidal anti-inflammatory drugs) such as aspirin, ibuprofen, or naproxen sodium; severe allergic reaction (e.g., anaphylaxis); burns; dehydration; blood clots in the veins and arteries in and around the kidneys; cholesterol deposits that block blood flow in the kidneys; glomerulonephritis; inflammation of the glomeruli; hemolytic uremic syndrome; lupus; use of medications such as chemotherapy drugs, antibiotics, or dyes used during imaging tests; scleroderma; thrombotic thrombocytopenic purpura; toxins including, for example, alcohol, heavy metals or cocaine; rhabdomyolysis; tumor lysis syndrome; bladder cancer; blood clots in the urinary tract; cervical cancer; colon cancer; enlarged prostate (e.g., benign prostatic hyperplasia); kidney stones; nerve damage involving the nerves that control the bladder; or prostate cancer.

Also provided herein is a method of treating fibrosis in a patient, such as administering to a patient an effective amount of a compound as described herein, administered in an amount effective to treat fibrosis in the patient. The fibrosis may result from trauma, as in wound healing, or as part of a disease or condition. The fibrosis may be pulmonary fibrosis, such as idiopathic pulmonary fibrosis. The fibrosis may be associated with, for example and without limitation, scleroderma; rheumatoid arthritis; Crohn's disease; ulcerative colitis; myelofibrosis; systemic lupus erythematosus; liver cirrhosis; non-alcoholic steatohepatitis; interstitial lung disease; kidney-, pancreas-, and heart-fibrosis; acne; and/or rosacea.

### Example 1—Compound Synthesis and Zebrafish Assay

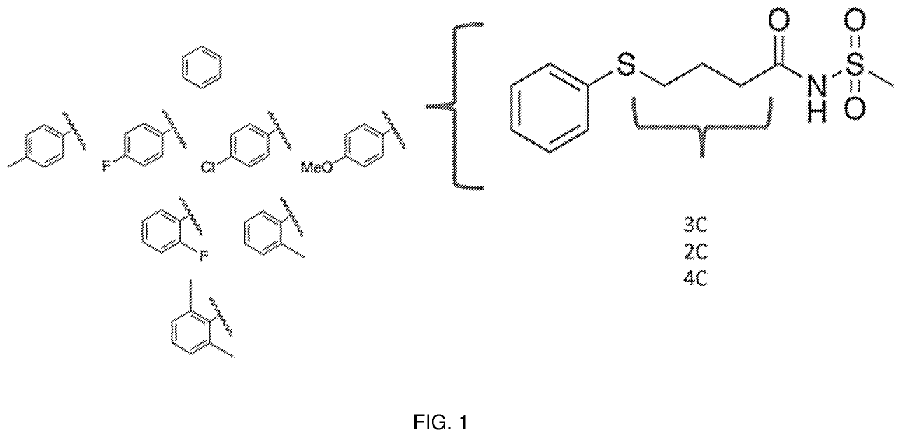

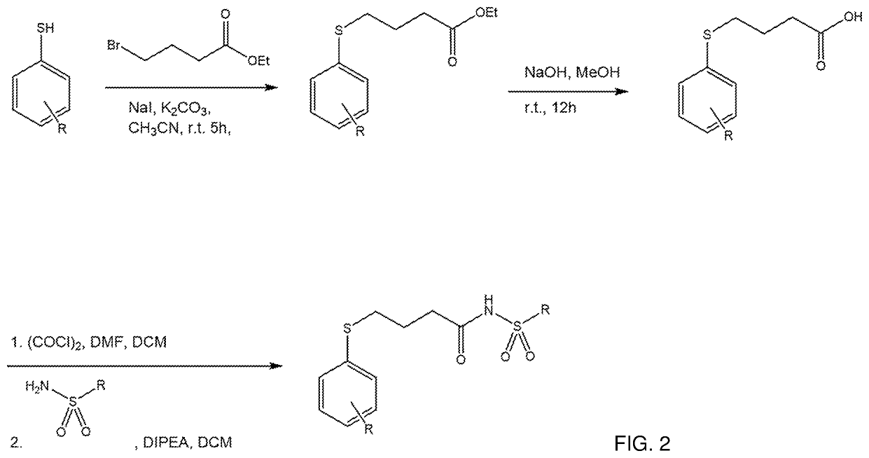

A library of compounds was prepared as shown in FIG. 1 using the synthesis scheme as depicted in FIG. 2.

Potential efficacy was tested in zebrafish assays, essentially as follows. Zebrafish were maintained as described (see, e.g., Cianciolo Cosentino, C., et al. (2013) Histone deacetylase inhibitor enhances recovery after AKI. J Am Soc Nephrol, 24 (6), 943-53 and Skrypnyk, N. I., et al. (2016) Delayed treatment with PTBA analogs reduces postinjury renal fibrosis after kidney injury. Am J Physiol Renal Physiol, 310 (8), F705-F716), and embryos from Pitt AB wildtype were used. Zebrafish larvae were injected with a single dose of gentamicin at 3 dpf with 7 ng of gentamicin as previously described. Prior to the gentamicin injection, 3 dpf zebrafish larvae were anesthetized in 0.2% tricaine/E3 medium (5 mM NaCl, 0.33 mM CaCl₂), 0.33 mM MgSO₄, and 0.17 mM KCl). Glass capillaries were pulled to produce microneedles and were aspirated with 10 ml of 7 ng/nL gentamicin solution diluted with filtered saline solution (Aspen Veterinary Resources, Cat No. 17861615). The larvae were injected with 1 nL gentamicin solution, delivered via the common cardinal vein. After injection, larvae were incubated in 50 μg/mL penicillin/streptomycin diluted in E3 medium. Test compounds were diluted in E3 medium containing 0.5% DMSO. Larvae were treated with either DMSO or test compounds (4 mM) from 2 days post-injection.

Data were analyzed as compounds that extend survival as measured by Kaplan-Meier estimator. UPHD 25 (compound previously shown to be protective in kidney injury) was used as a positive control and was statistically significant in all survival assays.

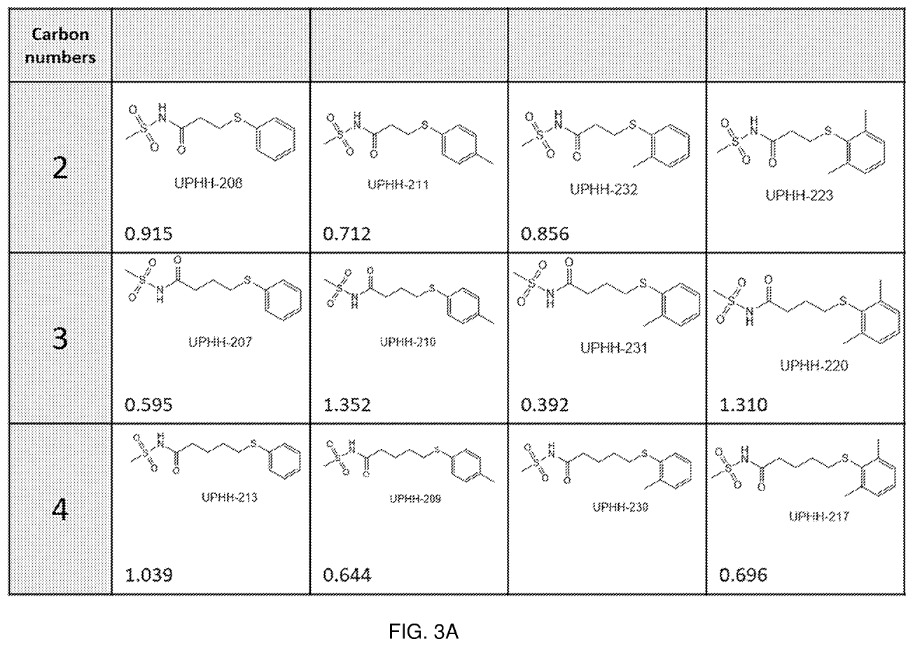

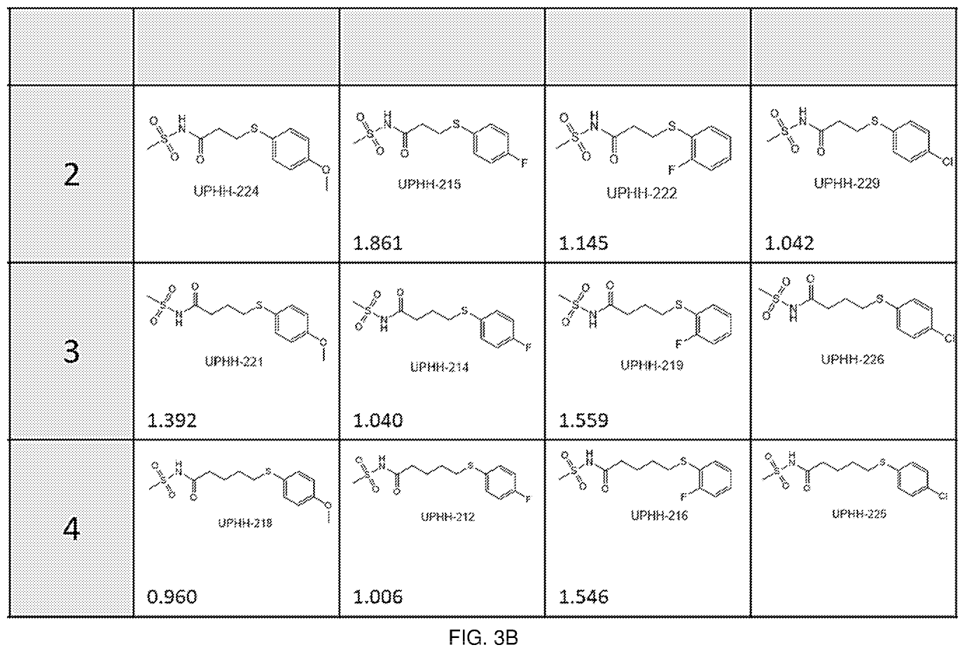

Table A provides preliminary calculated hazard ratios (n=1) for tested compounds (see also FIGS. 3A and 3B). A hazard ratio of less than one indicates that the zebrafish are more likely to survive with the compound. A hazard ratio of greater than one indicates that the zebrafish were more likely to die using the compound. As such, compounds 207 and 231 were the most promising under tested conditions. Due to the low number of tested subjects (n=1), those compounds with higher hazard ratios cannot be ruled out as promising candidates. For example, the effective therapeutic window may differ for certain compounds.

TABLE A

| | Hazard Ratio |
|---|---|
| UPHH # | (Mantel-Haenszel) |
| 207 | 0.595 |
| 208 | 0.915 |
| 210 | 1.352 |
| 211 | 0.712 |
| 212 | 1.006 |
| 213 | 1.039 |
| 214 | 1.040 |
| 215 | 1.861 |
| 216 | 1.546 |
| 217 | 0.696 |
| 218 | 0.960 |
| 219 | 1.559 |
| 220 | 1.310 |
| 221 | 1.392 |
| 229 | 1.042 |
| 231 | 0.392 |
| 232 | 0.856 |

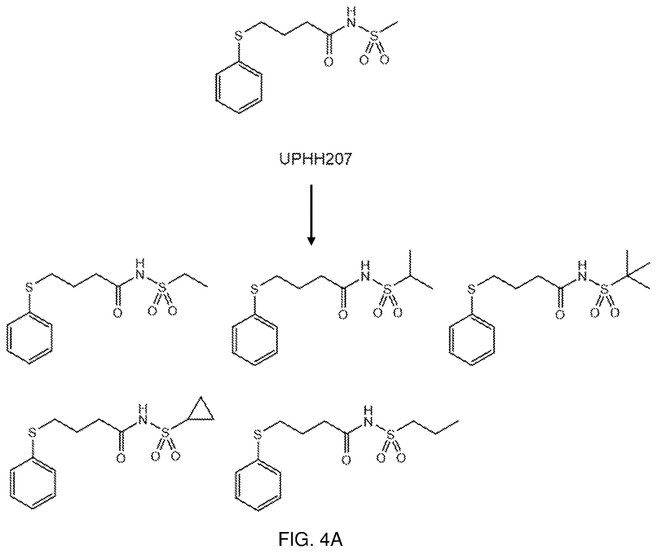

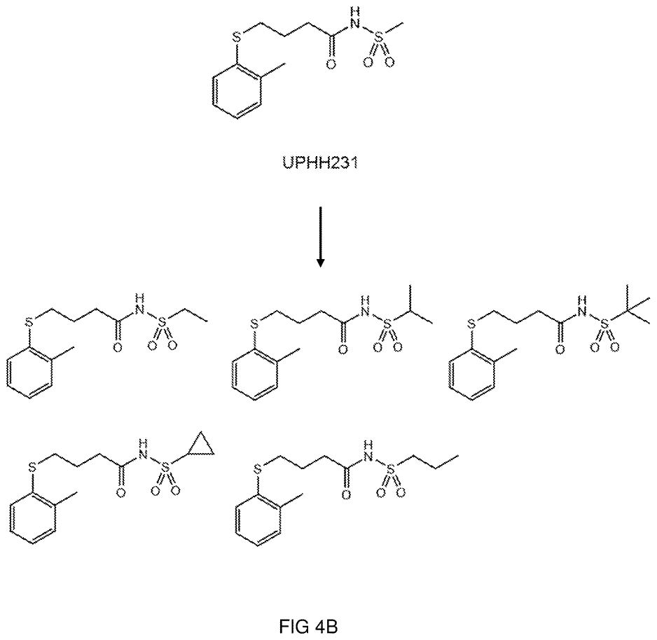

Based on the results shown in Table A, additional compounds based on compounds UPHH-207 and UPHH-231 may be formulated essentially as described in FIG. 2. Those compounds are depicted in FIGS. 4A and 4B.

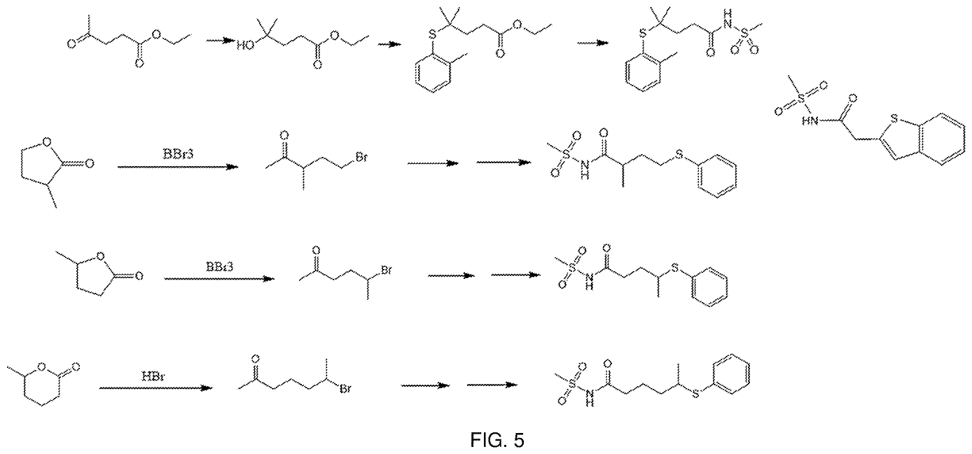

Additional derivatives with pendent methyl groups (branched alkylene groups for X in formula (I)), and/or a benzothiophene group may be synthesized as indicated in FIGS. 2 and 5.

Zebrafish embryos from Pitt AB wildtype were used. Zebrafish larvae were injected with a single dose of gentamicin at 3 days post-fertilization with 7 ng of gentamicin. Prior to the gentamicin injection, 3 days post-fertilization zebrafish larvae were anesthetized in 0.2% tricaine/E3 medium (5 mM NaCl, 0.33 mM CaCl₂), 0.33 mM MgSO₄, and 0.17 mM KCl). Glass capillaries were pulled to produce microneedles and were aspirated with 10 μL of 7 ng/nL gentamicin solution diluted with filtered saline solution (Aspen Veterinary Resources, Cat No. 17861615). The larvae were injected with 1 nL gentamicin solution, delivered via the common cardinal vein. After injection, larvae were incubated in 50 μg/mL penicillin/streptomycin diluted in E3 medium. Test compounds were diluted in E3 medium containing 0.5% DMSO. Larvae were treated with either DMSO, UPHD25, or UPHH207 (4 μM) from 2 days post-injection.

Both the control (UPHD25) and the test compound (UPHH207) show increase larval survival when compared to the injured, untreated control when assayed for days 5-11 days post-fertilization.

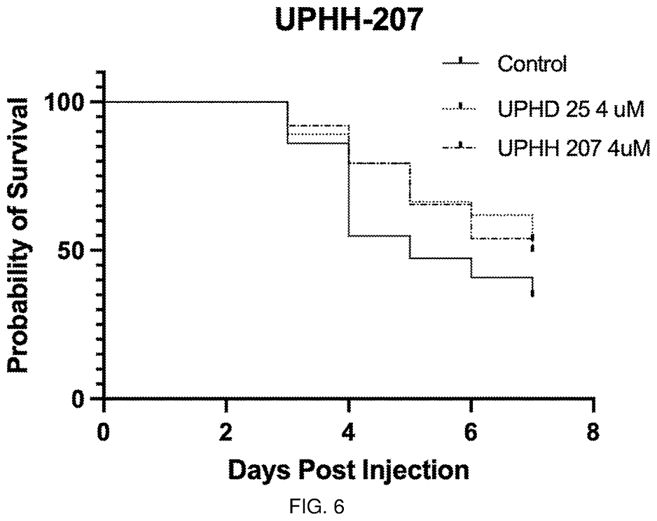

Further to the hazard ratios provided above, a Kaplan-Meier curve was generated for zebrafish treated with 4 μM of compound UPHH-207, essentially as described above. FIG. 6 illustrates that the efficacy of UPHH 207 is essentially equal to that of UPHD 25.

Effect of compound 207 on Hemin-injured Kidney Organoids: Injury, Inflammation, and Fibrosis. Organoid Assay. iPSCs were maintained on 10 cm cell culture dishes coated with Geltrex (Thermo Fisher) and mTeSR1 (Stemcell Technologies) medium. All experiments were performed with MANZ-2-2 iPSC line, generated in the Davidson laboratory. Kidney organoid assays were performed as described previously.33, Briefly after Dispase treatment iPSC clusters were suspended in medium composed of TeSR-E5 (Stemcell Technologies), 0.1% ITS-X, 1% CD Lipid concentrate (Gibco) and 0.25% polyvinyl alcohol, 1% penicillin/streptomycin (Gibco), and 2.5 ug/mL Plasmocin. On day 3 of the assay, embryoid bodies were transferred to the Stage II medium consisting of DMEM-low glucose, 10% KOSR (Thermo Fisher), 1% non-essential amino acids, 1% penicillin/streptomycin, 1% HEPES, 1% GlutaMAX, 0.25% poly-vinyl alcohol, 2.5 mg/mL Plasmocin. Hemin was made up in 0.1M NaOH. Day 14 organoids were washed thrice with DMEM-low glucose before being placed into protein-free medium (1:1 ratio of DMEM-low glucose and Hams F-12 Nutrient mixture, 1% HEPES, 1% penicillin/streptomycin (Gibco), and 2.5 ug/mL Plasmocin) containing Hemin in a 6-well ultra-low attachment plate. Hemin concentration was at 12.5 μM. Control well contained equivalent volume of 0.1 M NaOH as a vehicle control. All treatments were maintained for 48 hours.

Hemin treatment. Hemin (Millipore-Sigma) 10 mM stock concentration was resuspended in 0.1 M NaOH, sterile filtered and prepared fresh for every experiment. Day 14 organoids were washed thrice with DMEM-low glucose, then transferred into protein-free medium (1:1 ratio of DMEM-low glucose and Hams F-12 Nutrient mixture, 1×HEPES (to stabilize pH) 1% penicillin/streptomycin (Gibco), and 2.5 ug/mL Plasmocin) containing hemin in a 6-well ultra-low attachment (ULA) plate. The assay was then placed on a magnetic stir plate (2mag-USA) at 120 rpm, 25% power. Except where stated, hemin concentration was at 25 μM. Control well contained equivalent volume of 0.1 M NaOH as a vehicle control. All treatments were maintained for 48 hours, and thereafter washed thrice with Stage II medium (DMEM-low glucose, 10% knock-out serum replacement, 1% penicillin/streptomycin (Gibco), 1% Glutamax (Gibco), 1% HEPES, 1% MEM non-essential amino acids, 0.5% polyvinyl alcohol, 2.5 ug/mL Plasmocin) before proceeding to compound treatment. The pH of the control and hemin-containing media was tested following 48-hour incubation to exclude possibility of injury due to pH changes. The pH tested was 7.7 for control and 7.55 for hemin, within the normal range shown not to affect cellular apoptosis.

Compound treatments. Day 16 kidney organoids (post hemin treatment) were treated daily with UPHD25 compound. Stage II medium supplemented with 0.3% DMSO (Stage II-DMSO) was prepared. Firstly, a 2× stock solution of compound was prepared in Stage II-DMSO, and a calculated amount was added to each well to make up 1× working solution in a total of 3 mL volume, per well of a 6-well ULA plate. The plates were maintained on the magnetic stirrer at 25% power and 120 revolutions until fixation at day 26.

Histochemistry and analysis. Kidney organoids fixed in 4% paraformaldehyde and embedded in paraffin. Briefly, 6 μm thick sections were deparaffinized and heat-induced antigen retrieval performed using sodium citrate pH 6.0 buffer. Primary antibodies used were as follows; HAVCR1/KIM-1 (R&D Systems, AF1750), phosphor-histone H2A.X (ThermoFisher, 50-194-123), HMOX-1 (Santa Cruz, sc-136960), nitrotyrosine (Novus, NB110-96877), Collagen 1a1 (Abcam, ab138492). Fluorescently stained sections were imaged on a Zeiss LSM700 confocal microscope. COL1A1 imaging was done under the same settings established on the no-hemin control. Analysis was performed using ImageJ by combining the single channel images into one stack, subtracting the background (rolling ball radius of 50.0 pixels, sliding parapoloid), and applying a threshold. Threshold was determined based on the controls for each assay, and subsequently applied to the stack. Area of the threshold was then measured and calculated by division of the DAPI threshold area value. For analysis at least 3 assays were examined with >10 individual organoid sections per condition.

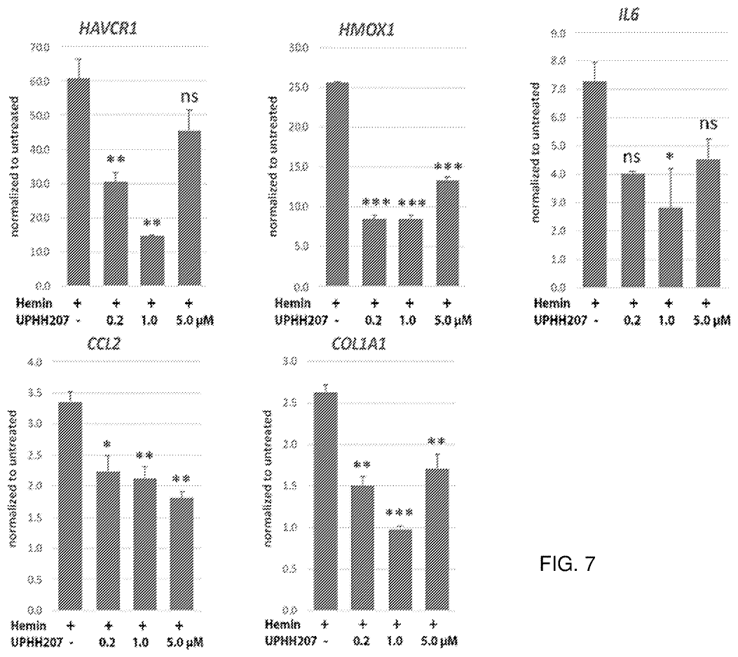

FIG. 7 shows qPCR quantification for injury markers (HAVCR1, HMOX1) and inflammation markers (IL6, CCL2, Col1A1) on organoids injured with 25 uM hemin and treated with different doses of UPHH207 as shown in the figure. All markers show at least one datapoint where there was a significant reduction in gene expression of either 0.05 (*) or 0.)1 (**).

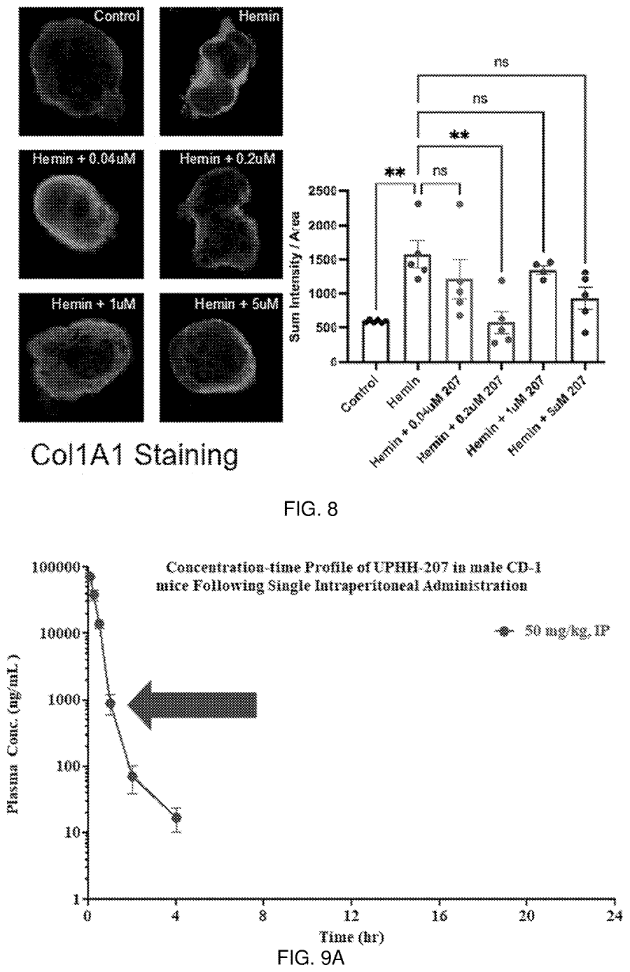

FIG. 8 shows antibody staining for the fibrosis marker collagen 1A1 using the Col1A1 Ab. Left panel shows organoid image with collagen deposition. The right panel shows quantification of images, each dot is an organoid. Overall, 0.2 uM of UPHH207 reduces the amount of collagen (fibrosis) significantly, compared to hemin injury, alone.

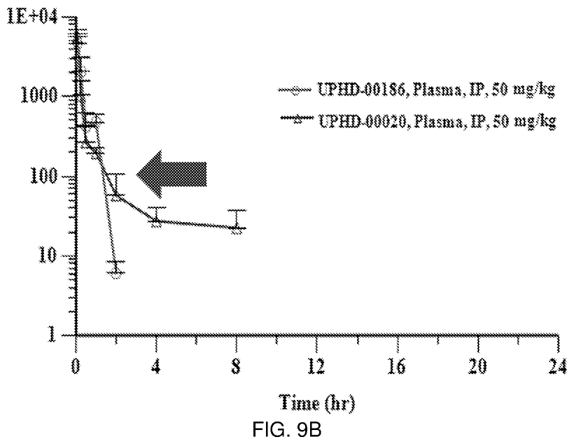

207 Exposure in Mice (ip). FIGS. 9A and 9B compare plasma concentrations-time profile (mean±SD) of UPHH-207 in male CD-1 mice following a single intraperitoneal administration (Dose: 50 mg/kg) In comparison to pro-drugs UPHH-186 and UPHH-20.

The objective of this study was to investigate the plasma pharmacokinetics of UPHH-207 in male CD-1 mice following single intraperitoneal administration at 50 mg/kg dose. Total nine mice (n=9) were used in this study with 3 mice/time points design. Animals were administered intraperitoneally with solution formulation of UPHH-207. The formulation vehicle was 5% v/v NMP, 5% v/v Solutol HS-15, 30% v/v PEG-400 and 60% v/v normal saline.

Blood samples (approximately 60 μL) were collected under light isoflurane anesthesia (Surgivet®) from retro orbital plexus from a set of three mice at Pre-dose, 0.08, 0.25, 0.5, 1, 2, 4, 8 and 24 hr. Immediately after blood collection, plasma was harvested by centrifugation at 4000 rpm, 10 min at 4° C. and samples were stored at −70±10° C. until bioanalysis. All samples were processed for analysis by protein precipitation method and analyzed with fit-for-purpose LC-MS/MS method (LLOQ=2.00 ng/mL for plasma). The plasma pharmacokinetic parameters were estimated using non-compartmental analysis tool of Phoenix® WinNonlin software (Ver 8.0) and parameters are summarized as follows: Route—IP; Dose (mg/kg)—50; Tₘₐₓ (hr)—0.08; Cmax (ng/mL) 73198.40; AUCₗₐₛₜ (hr*ng/mL)—23360.27; and T_{1/2} (hr)—0.56.

Following a single intraperitoneal administration of UPHH-207 in male CD-1 mice at 50 mg/kg dose, peak plasma concentration was observed at 0.08 hr, suggesting rapid absorption. The terminal elimination plasma half-life was 0.56 hr.

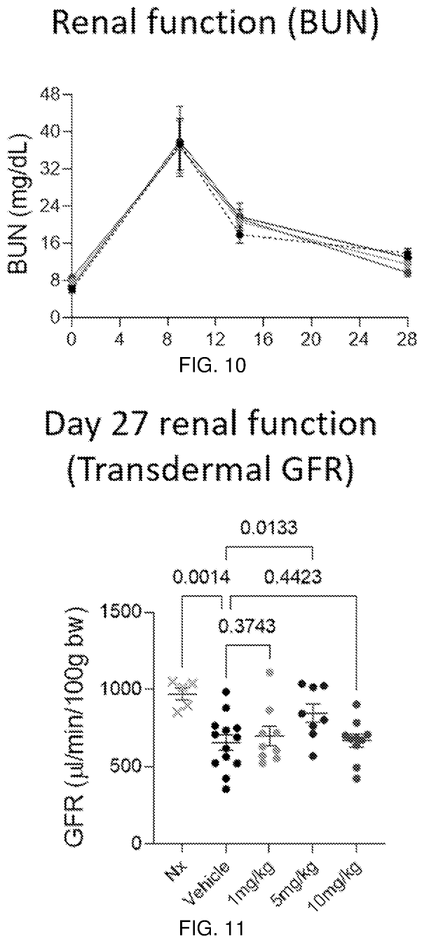

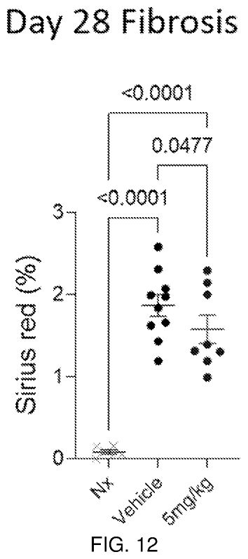

In an in vivo AKI model, unilateral injury was induced, and the compound was administered once a day for seven days. After 8 days, the uninjured contralateral kidney was removed, and renal function was evaluated with blood urea nitrogen (BUN) FIG. 10 and transdermal GFR (tGFR) FIG. 11 after the nephrectomy at different time points. BUN shows the expected increase 9 and 14 days after injury, but there is no significant difference between groups at 28 days. tGFR at 27 days after injury shows the anticipated reduction in GFR in the vehicle group compared to the nephrectomy control (Nx), with an increase in GFR in the 1 and 5 mg groups, which was significant at 5 mg. We also evaluated fibrosis by quantifying Sirius red staining of collagen in kidney sections 28 days after injury FIG. 12. As expected, there is a marked increase in fibrosis in the vehicle group, which was significantly decreased in mice treated with 5 mg/kg UPHH 207.

While the present invention has been described in terms of the above examples and detailed description, those of ordinary skill will understand that alterations may be made within the spirit of the invention. Accordingly, the above should not be considered limiting, and the scope of the invention is defined by the appended claims.

1. A compound is provided, having the structure (I): 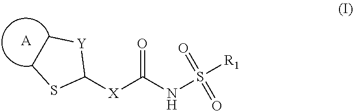
wherein:
A is a (C5-C7)(hetero)aryl ring and/or a phenyl bioisostere;
X is (C1-C7)divalent (hetero)alkyl;
Y is methylene (—CH₂—) or dimethylene (—CH₂—CH₂—) forming a 5- or 6-member ring with 0, 1, or 2 double bonds, or is not present; and
R₁ is (C1-C7)(hetero)alkyl, (C3-C7)(hetero)cycloalkyl, (C1-C3)(hetero)alkyl-(C3-C7)(hetero)cycloalkyl, (C1-C3)(hetero)alkyl-(C3-C7)aryl, (C1-C6)(hetero)alkoxyl, (C3-C7)(hetero)aryl-oxyl, or (hetero)aryl,
wherein A, X, or Y are, independently optionally substituted with one or more (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)(hetero)aryl-oxyl, (C3-C7)(hetero)aryl-amino, (C3-C7)(hetero)aryl-cyano, (C3-C7) (hetero)aryl-nitro, or (C3-C7) (hetero)aryl-halo groups,
or a pharmaceutically-acceptable salt thereof.

2. The compound of claim 1, having the structure (II): 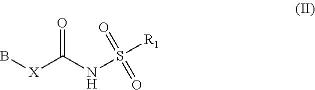
where:
B is: 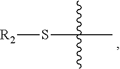
where R₂ is a C5-C7(hetero)aryl ring, or
a bicyclic heteroaryl group comprising a thiophene or thiopyran ring and a (C5-C7)aryl ring, such as a benzothiophenyl or benzothiopyranyl moiety;
X is (C1-C7)divalent (hetero)alkyl; and
R₁ is (C1-C7)(hetero)alkyl, (C3-C7)(hetero)cycloalkyl, (C1-C3)(hetero)alkyl-(C3-C7)(hetero)cycloalkyl, (C1-C3)(hetero)alkyl-(C3-C7)aryl, (C1-C6)(hetero)alkoxyl, (C3-C7)(hetero)aryl-oxyl, or halo, or a bioisostere of any of the preceding;
wherein B or X are, independently, optionally substituted with one or more (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7) (hetero)aryl-oxyl, (C3-C7) (hetero)aryl-amino, (C3-C7) (hetero)aryl-cyano, (C3-C7) (hetero)aryl-nitro, (C3-C7) (hetero)aryl-halo groups, and with the exception of X, halo, or a bioisostere of any of the preceding,
or a pharmaceutically-acceptable salt thereof.

3. The compound of claim 2, wherein the bicyclic heteroaryl group comprises a thiophene or thiopyran ring and a (C5-C7)aryl ring.

4. The compound of claim 3, wherein the bicyclic heteroaryl group is a benzothiophenyl or benzothiopyranyl moiety.

5. The compound of claim 4, wherein B is

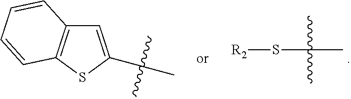

6. (canceled)

7. The compound of claim 5, wherein R₂ is phenyl, optionally substituted with one or more of (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, (C3-C7)aryl-amino, (C3-C7)aryl-cyano, (C3-C7)aryl-nitro, or (C3-C7)aryl-halo groups, or a bioisostere of any of the preceding.

8-9. (canceled)

10. The compound of claim 1, having the structure: 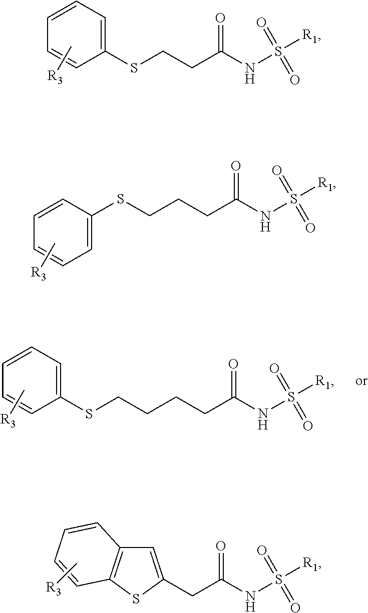
wherein R₃ is one or more of (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, (C3-C7)aryl-amino, (C3-C7)aryl-cyano, (C3-C7)aryl-nitro, or (C3-C7)aryl-halo groups,
or a pharmaceutically acceptable salt thereof.

11. The compound of claim 1, having the structure: 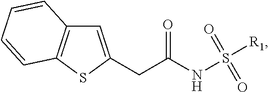
or a pharmaceutically acceptable salt thereof.

12. (canceled)

13. The compound of claim 1, having the structure: 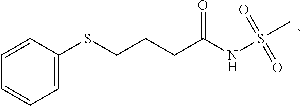 [SMILES: CS(=O)(=O)NC(=O)CCCSc1ccccc1]
or a pharmaceutically acceptable salt thereof.

14. The compound of claim 1, having the structure: 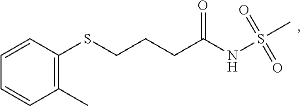 [SMILES: Cc1ccccc1SCCCC(=O)NS(C)(=O)=O]
or a pharmaceutically acceptable salt thereof.

15. The compound of the compound of claim 1, having the structure: 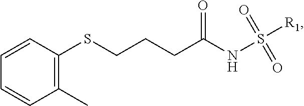
wherein R₁ is ethyl, cyclopropyl, n-propyl, isopropyl, or t-butyl, or a pharmaceutically acceptable salt thereof.

16. The compound of claim 1, having the structure: 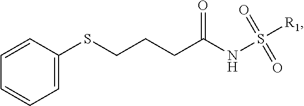
wherein R₁ is ethyl, cyclopropyl, n-propyl, isopropyl, or t-butyl.

17-18. (canceled)

19. The compound of claim 1, having the structure: 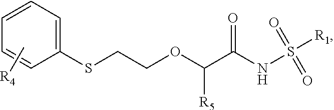
wherein, R₄ is one or more of H, (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, (C3-C7)aryl-amino, (C3-C7)aryl-cyano, (C3-C7)aryl-nitro, or (C3-C7)aryl-halo groups, and R₅ is (C1-C7)(hetero)alkyl or (C1-C7)(hetero)alkyl(hetero)aryl, optionally, either a bioisostere thereof, or substituted with one or more of (C1-C6)alkyl, (C3-C7)cycloalkyl, (C1-C3)alkyl-(C3-C7)cycloalkyl, (C1-C3)alkyl-(C3-C7)aryl, (C1-C6)alkoxyl, (C3-C7)aryl-oxyl, (C3-C7)aryl-amino, (C3-C7)aryl-cyano, (C3-C7)aryl-nitro, or (C3-C7)aryl-halo groups, or a pharmaceutically acceptable salt thereof.

20-21. (canceled)

22. The compound of claim 19, having the structure: 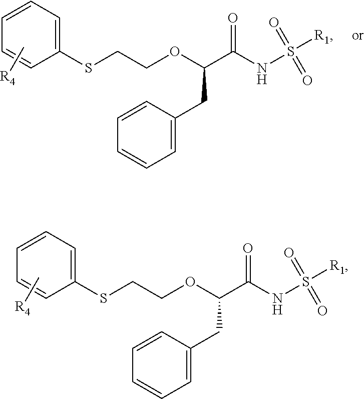
or a mixture thereof, and/or a pharmaceutically acceptable salt thereof.

23. (canceled)

24. The compound of claim 1 having a structure: 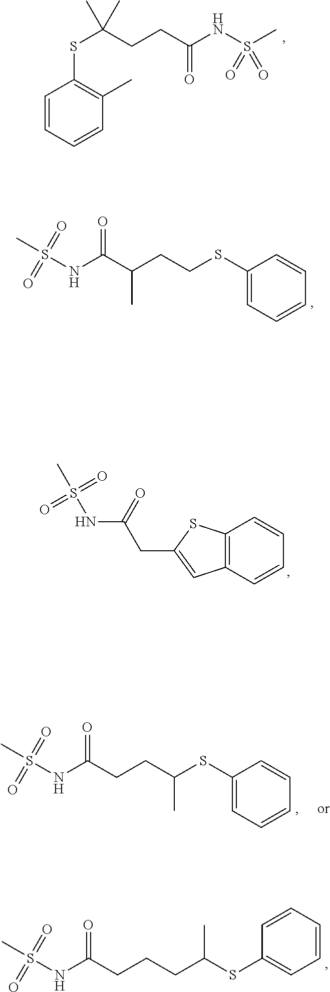
or a pharmaceutically acceptable salt thereof.

25. A composition comprising the compound claim 1, and a pharmaceutically-acceptable excipient.

26. The composition of claim 25, comprising a compound having the structure: 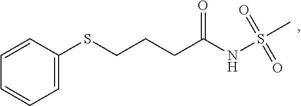 [SMILES: CS(=O)(=O)NC(=O)CCCSc1ccccc1]
or a pharmaceutically acceptable salt thereof; or 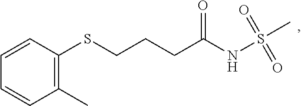 [SMILES: Cc1ccccc1SCCCC(=O)NS(C)(=O)=O]
or a pharmaceutically acceptable salt thereof.

27. A method of treating kidney injury in a patient, comprising administering to the patient a compound as claimed in claim 1, in an amount effective to treat kidney injury.

28. The method of claim 27, wherein the kidney injury is acute kidney injury.

29-30. (canceled)

31. A method of treating fibrosis, in a patient, comprising administering to the patient a compound as claimed in claim 1 in an amount and dosage regimen effective to treat fibrosis in the patient.

32-33. (canceled)
